(19) United States
(12) Patent Application Publication
(10) Pub. No.: US 2005/0019454 A1
(43) Pub. Date: Jan. 27, 2005

(54) Eating quality grade

(76) Inventor: Hamish Alexander Nigel Kennedy, Mount Eden (NZ)

Correspondence Address: KOPPEL, JACOBS, PATRICK & HEYBL, 555 ST. CHARLES DRIVE, SUITE 107, THOUSAND OAKS, CA, 91360, US

(21) Appl. No.: 10/275,363

(22) PCT Filed: May 2, 2001
(86) PCT No.: PCT/NZ01/00070

(30) Foreign Application Priority Data
May 5, 2000 (NZ) 504370

(51) Int. Cl. G01N 33/02

(52) U.S. Cl. 426/231

(57) ABSTRACT

An eating quality grade for a food product includes the identification of one or more internal characteristics of the food product such as sweetness, brix, firmness and/or acidity in determining which of those internal characteristics provides for a defined grade of eating quality. The correlation of eating quality and internal characteristics are referenced so that having determined the internal characteristics of a particular fruit this will enable the grade from the eating quality grade to be allocated. It is envisaged that a common grade could be utilised for a variety of food products.

### BACKGROUND OF THE INVENTION

The present invention relates to a method and/or apparatus for identifying the eating quality of food products, particularly but not exclusively, fruit. The invention will, however, be described in respect of its use with fruit, but it will be understood by those involved in food technology that the invention has application across the whole range of food products.

To the present time, various proposals have been made for grading specific food products and in achieving this, various types of technology have been proposed for identifying particular qualities of a food product such as its weight, firmness, colour, sugar content and the like. In my International Application PCT/NZ99/00150, there is described a food product identification system, which may utilise electromagnetic energy in the near infra red (NIR) frequency range, while in my International Application PCT/NZ99/00151, there is described a quality sorting of food products. Again, this may use NIR Spectroscopy in conjunction with a shield.

These and other technologies have not however allowed the grading of a fruit with a particular grade for its taste quality depending on its internal characteristics, nor a common grade across a variety of food products.

### OBJECTS OF THE INVENTION

It is, thus, an object of the present invention to provide a method and/or apparatus, which can provide a grading of the taste of a food product depending on one or more of its internal qualities. For the sake of simplicity, this grading will be referred to as the “eating quality grade”.

Further objects of this invention will become apparent from the following description.

### STATEMENTS OF INVENTION

According to one aspect of the present invention, there is, thus, provided a method of providing a common eating quality grade for a variety of food products, including:
(i) identifying one or more internal characteristics of each of the food products which determine its eating quality;
(ii) providing one or more common grades of at least acceptable eating quality of the food products and correlating the grade(s) with the internal characteristic(s);
(iii) recording the one or more internal characteristics to provide a reference of the characteristic(s) required for each grade for each food product;
(iv) measuring the internal characteristic(s) of food products, or at least a representative sample of them, and grading the food products according to the reference.

Preferably the measuring of the internal characteristic(s) may include moving a plurality of the food products relative to a sensing means or vice versa, which sensing means will sense the one or more characteristics for each of the food products

Preferably data from the sensing means may be used to determine the grade for a particular food product.

According to a further aspect of the present invention a method of providing an eating quality grade for a food product includes:
(i) identifying one or more internal characteristics of a food product which determine its eating quality;
(ii) providing one or more grades of at least acceptable eating quality and correlating the grade(s) with the one or more internal characteristics;
(iii) measuring the one or more internal characteristics required for the or each grade to provide a reference of the characteristic(s) required for each grade; and
(iv) measuring, according to the reference, the internal characteristic(s) of the food product, or at least a representative sample of it, and grading the food product according to the reference.

According to a further aspect of the present invention, there is provided a food product grading means, including:
(i) a sensing means, which can sense one or more internal characteristic(s) of a food product, which characteristics to be utilised in calculating the grade of desired taste characteristics for a particular food product;
(ii) means to enable a plurality of the food products to move relative to the sensing means or vice versa;
(iii) a reference means cross referencing the internal characteristic(s) with one or more grades of at least acceptable eating quality;
(iv) processing means to utilise the data from the sensing means in order to determine the grade for a particular food product.

Preferably, the internal characteristic of the fruit includes its sweetness or brix.

Preferably, the internal characteristic may include firmness and/or acidity.

Preferably there is provided labelling of the products to identify a particular grade.

Further aspects of this invention, which should be considered in all its novel aspects will become apparent from the following description, given by way of example of possible embodiments of the invention.

### BRIEF DESCRIPTION OF THE DRAWINGS

The description will now be provided with reference to the drawings in which:

FIG. 1: Shows a flow diagram of a grading operation for one possible embodiment of the invention;

FIG. 2: Shows a flow diagram of a further grading operation according to one possible embodiment of the invention.

### DETAILED DESCRIPTION OF PREFERRED EMBODIMENTS

The characteristics of a food product such as fruit, which are preferred by the commercial producers or retailers may be quite distinct from those most sought after by the ultimate consumers. The commercial producer may consider the external appearance of the fruit of critical importance, with other factors such as yield and durability in storage and shipping also being important.

On the other hand, the consumer would of course be wanting the best taste or eating quality. The eating quality as well as taste can encompass sweetness, aromas, texture, freshness, crispness, etc.

The internal eating quality is, however, not apparent to the consumer when purchasing the fruit, so a selection is generally made again on external appearance, which can be deceptive.

In the selling of fruit, it is inevitably graded prior to sale and inevitably the more desirable fruit is sold at the higher price. This grading has, in the past, been dominated by the visible features of size, shape, colour and external appearance.

However, technology, is now available, such as that described in my aforementioned patent specifications, which can determine the internal or eating quality of a food product such as fruit.

Depending on the type of fruit, different eating qualities may apply, such as dry matter, brix, acids, brix-acid ratio, oils, firmness, texture, etc.

It will be appreciated that different internal characteristics may provide better eating quality depending on the fruit concerned, and by way of example only, a peach may require a brix of at least twelve, kiwifruit may require a brix of at least fourteen, and mandarins may require a brix of at least thirteen with a particular acid content. The above values are merely very approximate examples.

To assist the selection of a food product such as a fruit based on its eating quality grade, the present invention in one embodiment provides a grading which is common across a variety of fruit so that a consumer will know if a peach or a kiwifruit is selected having an eating quality grade “X”, that it will have a particular eating quality, although the calculation of that grade from the internal characteristics of the fruit which have been assessed will vary between fruit.

The eating quality grade can, of course, be formulated in any number of ways and could, for example, utilise the numbers one to ten or the alphabet.

In one example, however, a graduated scale of one to nine could be used, with one being poor and nine being excellent.

Also, a mixture of grades could be used such as “AB” could indicate a sweetness A and a storage capacity B.

The internal properties which make for a good eating quality will then be determined and these may, of course, vary between different markets, and even may have regional or ethnic variations within the same market, for example, Northern Europeans may prefer more acid fruit to those from Southern Europe.

The invention then provides a reference which may be a matrix or algorithm for example which will, for each type of fruit, correlate eating quality and the selected internal properties. However in a preferred embodiment, the reference grade will remain the same, in that a grade “X” peach will represent the equivalent excellent eating quality as would a grade “X” kiwifruit. The fruit and other produce required to be graded may be moved relative to the one or more sensing means or vice versa, which non-destructively will sense the internal characteristics. The technology used by the sensor means may, as mentioned above, include NIR spectroscopy and/or acoustic resonance, deformation, nuclear magnetic resonance, microwave, or the like.

The data from the one or more sensing means will then be matched against the grade for the particular fruit for the eating quality grade to be calculated. The fruit can then be sorted and labelled according to that eating quality grade. It is envisaged that a neural network, or the like could be used instead of a matrix type of reference.

It is seen, therefore, that individual fruits, or other food products can be graded on line, such as on a conveyor type machine and with a common eating quality grade being applied to a variety of fruit, grading them by their internal eating quality and not by their external appearance. However, extrinsic factors such as size, weight, bruising, or the like, may be taken into account, for example, in discarding any fruit which may be excellent internally, but may suffer from some abnormality or damage.

Also, it is envisaged that external characteristics could be taken into account in the designation of the grade either in a combined designator, say AB, or a single designator.

Also, it is envisaged that the reference grading could accommodate a situation where an average of a particular characteristic for a particular batch or line of products could lower or higher the threshold of an internal characteristic, such as brix, which is being measured.

In providing the eating quality grade of the present invention, it is envisaged that remote purchasing will be made much more acceptable, e.g. over the internet.

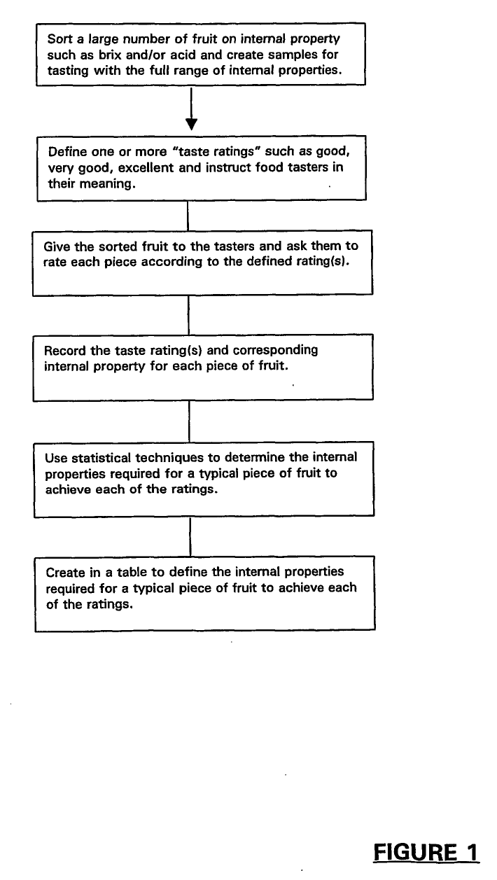

Referring now to the accompanying drawings, firstly FIG. 1 shows one possible embodiment of the invention providing a reference table or the like for the gradation of eating quality grade for a particular fruit. As shown in FIG. 1 the present invention in its application to fruit will first provide for the sorting of a large number of the fruit dependent on one or more internal properties such as brix and/or acid.

One or more “taste ratings” will then be specified for the fruit which will provide the previously mentioned eating quality grade.

The sorted fruit will then be weighted as far as that eating quality grade is concerned and those ratings will then be correlated against the previously identified internal property or properties.

It will then be determined which internal property or properties provide the basis of the gradation of eating quality grade so that this can then be tabulated or otherwise made into a reference, providing a correlation between the one or more internal properties and the ratings.

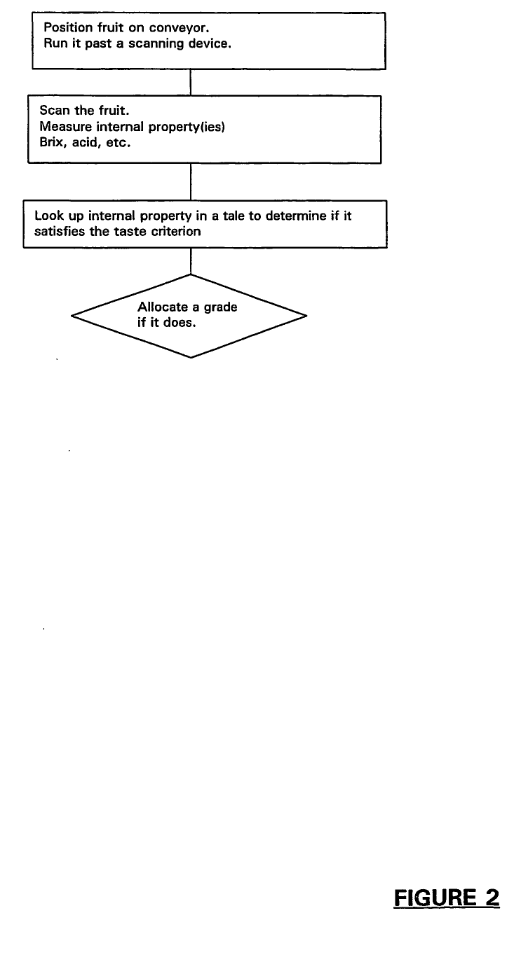

Then as shown in FIG. 2, in the use of the invention and in one particular embodiment, fruit required to be graded may be run past a scanning device so that the one or more internal properties can be measured such as brix or acid. The internal property or properties of a particular fruit can then be cross-referenced to the table to see what grade the fruit has achieved or whether it is deficient in not reaching even a minimum level for gradation purposes.

It is envisaged that a labelling means may then allocate an appropriate grading label to the fruit dependent on its grading if not rejected.

In the commercial setting it may not be necessary to grade all food product from a particular batch. Depending on the particular product and its source it may be appropriate to test only a certain, representative, sample of a batch and if that is of a sufficient quality, on average, then the whole batch could be assumed to reach the required standard. For fruit grading this would enable processing equipment to have only one lane of a multi-lane machine equipped with the required scanner(s). Alternatively, scanning could be carried out on a separate sampling machine.

While the invention has been described with particular reference to fruit it will be appreciated that it is not limited to food of that type and the method may be used to grade other foods, for example meats for which important characteristics may include tenderness and fat content.

Where, in the foregoing description, reference has been made to specific components or integers of the invention having known equivalents then such equivalents are herein incorporated as if individually set forth.

Although this invention has been described by way of example and with reference to possible embodiments thereof, it is to be understood that modifications or improvements may be made thereto without departing from the scope of the invention as defined in the appended claims.

1. A method of providing an eating quality grade for a food product includes:
identifying one or more internal characteristics of a food product which determine its eating quality;
providing one or more grades of at least acceptable eating quality and correlating the grade(s) with the one or more internal characteristics;
measuring the one or more internal characteristics required for the or each grade to provide a reference of the characteristic(s) required for each grade; and
measuring, according to the reference, the internal characteristic(s) of the food product, or at least a representative sample of it, and grading the food product according to the reference.

2. A method as claimed in claim 1 wherein the one or more internal characteristics includes its sweetness or brix.

3. A method as claimed in claim 1 wherein the one or more internal characteristics includes firmness.

4. A method as claimed in claim 1 in which the one or more internal characteristics includes acidity.

5. A method of providing a common eating quality grade for a variety of food products, including:
identifying one or more internal characteristics of each of the food products which determine its eating quality;
providing one or more common grades of at least acceptable eating quality of the food products and correlating the grade(s) with the internal characteristic(s);
recording the one or more internal characteristics to provide a reference of the characteristic(s) required for each grade for each food product;
measuring the internal characteristic(s) of food products, or at least a representative sample of them, and grading the food products according to the reference.

6. A food product grading means including:
sensing means, which can sense one or more internal characteristic(s) of a food product, which characteristics to be utilised in calculating the grade of desired taste characteristics for a particular food product;
means to enable a plurality of the food products to move relative to the sensing means or vice versa;
reference means cross referencing the internal characteristic(s) with one or more grades of at least acceptable eating quality;
processing means to utilise the data from the sensing means in order to determine the grade for a particular food product.

7. A food product grading means in which the sensing means detects sweetness, brix, firmness and/or acidity.

9. A method as claimed in claim 2 in which the one or more internal characteristics includes firmness.

10. A method as claimed in claim 2 in which the one or more internal characteristics includes acidity.

11. A method as claimed in claim 3 in which the one or more internal characteristics includes acidity.
